(19) United States
(12) Patent Application Publication
(10) Pub. No.: US 2023/0064377 A1
(43) Pub. Date: Mar. 2, 2023

(54) PREPARATION METHOD OF A HIGH-OPTICAL-PURITY CHIRAL OXO-AZA-CYCLOALKANE COMPOUND

(71) Applicant: XIN FA PHARMACEUTICAL CO., LTD., Dongying (CN)

(72) Inventors: Xianlong JIANG, Dongying (CN); Mingfeng ZHANG, Dongying (CN); Yuxin QI, Dongying (CN)

(21) Appl. No.: 17/758,384

(22) PCT Filed: Oct. 20, 2020
(86) PCT No.: PCT/CN2020/122111

§ 371 Date: Jul. 5, 2022

(30) Foreign Application Priority Data
Jan. 20, 2020 (CN) 202010064144.9

(51) Int. Cl. C07D 211/60; C07D 207/16

(52) U.S. Cl. CPC C07D 211/60; C07D 207/16; C07B 2200/07

(57) ABSTRACT

A preparation method of a high-optical-purity chiral oxo-aza-cycloalkane compound comprises the steps of: (R or S)N-substituent P-2-alkoxycarbonylalkyliminodiacetic acid diester as the raw material is cyclized in the presence of a solvent and a cyclization reagent (lewis acid-lewis base) to obtain high-optical-purity N-substituent P -2- (R or S)- substituent G oxo-aza-cycloalkane or the salt thereof through thermal decarboxylation or hydrolysis decarboxylation reaction; the specific type of cyclization reagent used in the present invention is conductive to the preparation of the high-optical-purity target product, easily recoverable, with reduced waste liquid discharge, green, and environment-friendly, which contributes to green industrial production.

### FIELD OF THE INVENTION

The present invention relates to a preparation method of a high-optical-purity chiral oxo-aza-cycloalkane compound and particularly to a preparation method of a high-optical-purity N-substituent P -2- (R or S)- substituent G oxo-aza-cycloalkane or the salt thereof. It pertains to the technical field of fine chemistry.

### BACKGROUND OF THE INVENTION

The high-optical-purity N-substituent P -2- (R or S)- substituent G oxo-aza-cycloalkane or the salt thereof is an important intermediate. Based on the nitrogen atom, the chiral acid or the derivative thereof, and the heterocyclic carbanyl group contained in it, multiple compounds can be derived from it and then used in the research and development of medicines, pesticides and the like.

Optically pure N-substituent P -2- (R or S)- substituent G oxo-aza-cycloalkane (I) or the salt thereof, wherein n is 1, 2, or 3; specially, when n is 1, the compound of formula I is optically pure N- substituent P -2- (R or S) - substituent G -4-oxo-tetrahydropyrrole or the salt thereof; when n is 2, the compound of formula I is optically pure N-substituent P-2-(R or S)-substituent G-5-oxo-piperidine or the salt thereof; when n is 3, the compound of formula I is optically pure N-substituent P-2-(R or S)-substituent G-6-oxoheterocycloheptane or the salt thereof. The compound of formula I has the structure as shown below:

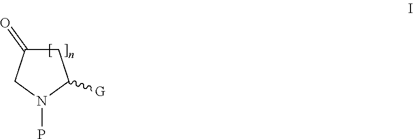

Wherein, in the compound of formula I, the substituent P is benzyl, o-methoxybenzyl, m-methoxybenzyl, p-methoxybenzyl, 2,4-dimethoxybenzyl, o-methylbenzyl, m-methylbenzyl, p-methylbenzyl, o-chlorobenzyl, p-chlorobenzyl, m-chlorobenzyl, benzoyl, methoxycarbonyl, tert-butoxycarbonyl, or benzyloxycarbonyl; substituent G is COOH or COOR, wherein R is methyl, ethyl, n-propyl, isopropyl, tert-butyl, n-butyl, sec-butyl, or benzyl.

The salt of the compound of the formula I is specifically the hydrochloride, hydrobromide, hydriodate, sulphate, phosphate, or acetate salt of the compound of formula I.

### SUMMARY OF THE INVENTION

To address the drawbacks in the prior arts, the present invention provides a preparation method of a high-optical-purity chiral oxo-aza-cycloalkane compound and particularly a preparation method a high-optical-purity N-substituent P -2- (R or S)- substituent G oxo-aza-cycloalkane or the salt thereof.

Description of terms:

Compound of formula II: (R or S)N-substituent P-2-alkoxycarbonylalkyliminodiacetic acid diester;

Compound of formula I: N-substituent P -2- (R or S)- substituent G oxo-aza-cycloalkane.

Chiral oxo-aza-cycloalkane compound: The compound of formula I or the salt thereof.

The numbering of the compounds in the description is completely consistent with the numbering of their structural formulas, and they have same references.

A technical solution of the present invention is provided below:

A preparation method of chiral oxo-aza-cycloalkane compound, comprising the steps of:

A compound of formula II is cyclized and then decarboxylated to obtain a compound of formula I or the salt thereof;

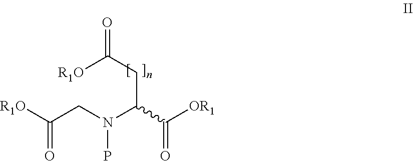

Wherein, the substituent R₁ in the compound of formula II is methyl, ethyl, n-propyl, isopropyl, tert-butyl, n-butyl, sec-butyl or benzyl; substituent P is benzyl, o-methoxybenzyl, m-methoxybenzyl, p-methoxybenzyl, 2,4-dimethoxybenzyl, o-methylbenzyl, m-methylbenzyl, p-methylbenzyl, o-chlorobenzyl, p-chlorobenzyl, m-chlorobenzyl, benzoyl, methoxycarbonyl, tert-butoxycarbonyl, or benzyloxycarbonyl; n is 1, 2, or 3.

Preferably according to the present invention, the said compound of formula II isN-benzyl-2S-methoxycarbonyl methyl dimethyl iminodiacetate, N-benzyl-2S-ethoxycarbonyl methyl diethyl iminodiacetate, N-benzyl-2S-methoxycarbonyl ethyl dimethyl iminodiacetate, N-benzyl-2S-ethoxycarbonyl ethyl diethyl iminodiacetate, N-benzyl-2R-methoxycarbonyl ethyl dimethyl iminodiacetate, N-benzyl-2R-ethoxycarbonyl ethyl diethyl iminodiacetate, N-benzyl-2S-methoxycarbonyl propyl dimethyl iminodiacetate, N-benzyl-2S-ethoxycarbonyl propyl diethyl iminodiacetate, N-p-chlorobenzyl-2S-Isopropoxycarbonyl ethyl diethyl iminodiisopropyladipate, or N-benzyloxycarbonyl-2S-ethoxycarbonyl methyl diethyl iminodiacetate.

Preferably according to the present invention, the compound of formula II is cyclized in a solvent under the action of a cyclization reagent.

Preferably, the said solvent is tetrahydrofuran, 2-methyl tetrahydrofuran, 1,4-dioxane, ethylene glycol dimethyl ether, methyl tert-butyl ether, methoxy-cyclopentane, dichloromethane, chloroform, 1,2-dichloroethane, normal hexane, n-heptane, or methylbenzene, or a combination of two or more thereof; the mass ratio between the said solvent and the compound of formula II is (3-10):1.

Preferably, the said cyclization reagent is the mixture of a lewis acid and a lewis base; the said lewis acid is aluminium trichloride, boron trifluoride, titanium tetrachloride, or stannic chloride, and the said lewis base is trimethylamine, triethylamine, tri-n-butylamine, diisopropylethylamine, or pyridine, or a combination of two or more thereof; the molar ratio among the said lewis acid, lewis base, and compound of formula II is (1.0-4.0): (1.0-4.0):1.

Preferably, the temperature of the said cyclization reaction is -60-50° C.; more preferably, the temperature of the said cyclization reaction is -30-20° C.; most preferably, the temperature of the said cyclization reaction is 0-15° C. The duration of the said cyclization reaction is 0.5-5 hours; more preferably, the duration of the said cyclization reaction is 1-3 hours. Too high a cyclization reaction temperature will cause polymerization reaction of the raw materials, resulting in by-products and reducing the optical purity and yield of the target product.

Preferably according to the present invention, after the cyclization reaction, the compound of formula II proceeds directly to the next step without separation.

Preferably according to the present invention, the said decarboxylation reaction is to obtain the compound of formula I through thermal decarboxylation in the presence of N, N-dimethyl formamide (DMF) and lithium chloride, or obtain a salt of the compound of formula I through hydrolysis decarboxylation in the presence of an acid.

Preferably, the mass ratio between the said DMF and the compound of formula II is (2.0-10.0): 1, and that between the said lithium chloride and the compound of formula II is (2.0-20.0%):1; more preferably, the mass ratio between the said DMF and the compound of formula II is (2.0-5.0): 1, and that between the said lithium chloride and the compound of formula II is (5.0-10.0%): 1.

Preferably, the temperature of the said thermal decarboxylation reaction is 100-180° C.; more preferably, the temperature of the said thermal decarboxylation reaction is 130-150° C. The duration of the said thermal decarboxylation reaction is 1-10 hours; more preferably, the duration of the said thermal decarboxylation reaction is 2-5 hours. Too high a thermal decarboxylation temperature will produce a large quantity of thermal decomposition byproducts.

Preferably, the said acid is hydrochloric acid, hydrobromic acid, hydroiodic acid, acetic acid, sulfuric acid, or phosphoric acid, or a combination of any two or more thereof; more preferably, the said acid is hydrochloric acid; the molar ratio between the said acid and the compound of formulaII is (1.0-10.0):1; more preferably, the molar ratio between the said acid and the compound of formulaII is (4.0-10.0):1.

Preferably, the temperature of the said hydrolysis decarboxylation reaction is 30-110° C.; more preferably, the temperature of the said hydrolysis decarboxylation reaction is 60-100° C.; most preferably, the temperature of the said hydrolysis decarboxylation reaction is 60-80° C. The duration of the said hydrolysis decarboxylation reaction is 1-10 hours; more preferably, the duration of the said hydrolysis decarboxylation reaction 1-5 hours. Too high a reaction temperature will produce a large quantity of byproducts.

Preferably according to the present invention, the salt of the compound of formula I is the hydrochloride, hydrobromide, hydriodate, sulphate, phosphate, or acetate of the compound of formula I.

The present invention provides a reaction route as shown below:

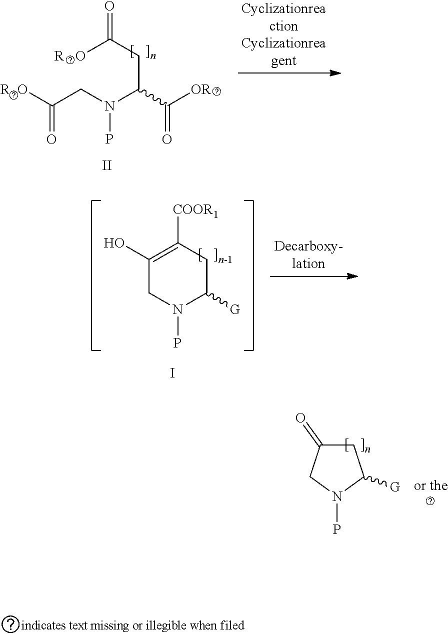

Wherein, the substituent R₁ in the structural formula of the compound of formula II is methyl, ethyl, n-propyl, isopropyl, tert-butyl, n-butyl, sec-butyl, or benzyl; the substituent P is benzyl, o-methoxybenzyl, m-methoxybenzyl, p-methoxybenzyl, 2,4-dimethoxybenzyl, o-methylbenzyl, m-methylbenzyl, p-methylbenzyl, o-chlorobenzyl, p-chlorobenzyl, m-chlorobenzyl, benzoyl, methoxycarbonyl, tert-butoxycarbonyl, or benzyloxycarbonyl; n is 1, 2 or 3. In the intermediate obtained through the cyclization reaction of the compound of formula II, the n, substituent P, and substituent R₁ have the same meanings as those in the structural formula of the compound of formula II; when n is 1, it means that the double-bonded carbon linked with COOR₁ is directly bonded to the carbon linked with the substituent G; the substituent G is COOH or COOR, wherein R is methyl, ethyl, n-propyl, isopropyl, tert-butyl, n-butyl, sec-butyl, or benzyl and the substituent R has the same meaning as the substituent R₁ in the structural formula of the compound of formula II. In the structural formula of the compound of formula I, the substituent P, n, and substituent G have the same meanings as those in the abovesaid intermediate.

The technical characteristics and beneficial effects of the invention are as follows:

1. The present invention provides a preparation method of a high-optical-purity N-substituent P -2- (R or S)- substituent G oxo-aza-cycloalkane or the salt thereof. In the present invention, (R or S)N-substituent P-2-alkoxycarbonylalkyliminodiacetic acid diester as the raw material is cyclized in the presence of a solvent and a cyclization reagent (lewis acid-lewis base) to obtain N-substituent P-2-(R or S)-substituent G-(n+1)-carbalkoxy oxo-aza-cycloalkane. Without separation, the resulting product is then subjected to a thermal decarboxylation or hydrolysis decarboxylation reaction to obtain high-optical-purity N-substituent P -2- (R or S)- substituent G oxo-aza-cycloalkane or the salt thereof.

2. Compared with the prior arts, the present invention uses inexpensive and easily accessible raw material, which reduces the cost; the reaction conditions are easily controllable and achievable. The specific type of cyclization reagent used in the present invention is conductive to the preparation of the high-optical-purity target product, easily recoverable, green, and environment-friendly, which contributes to green industrial production. The raw material and products involved in the present invention are with stable chiral structures, and the resulting target product has a high optical purity and a high yield. With the chiral carbon atom in the ortho position of the ester group, the raw material (compound of formula II) used in the present invention can be racemized by the carboanion when condensed under conventional strong base conditions, so the chiral carbon atom can keep inert under the reaction conditions to maintain chirality, presenting a good reaction selectivity. The preparation method of a high-optical-purity N-substituent P -2- (R or S)- substituent G oxo-aza-cycloalkane or the salt thereof in the present invention lays the foundation for studying the biological activity of a series of chiral oxo-aza-cycloalkane derivatives.

### BRIEF DESCRIPTION OF THE DRAWINGS

FIG. 1 provides the NMR (Nuclear Magnetic Resonance) hydrogen spectrogram of the target product obtained from the Embodiment 1 of the present invention.

FIG. 2 provides the NMR (Nuclear Magnetic Resonance) carbon spectrogram of the target product obtained from the Embodiment 1 of the present invention.

FIG. 3 provides the normal-phase HPLC chromatogram of the target product obtained from the Embodiment 1 of the present invention.

FIG. 4 provides the normal-phase HPLC chromatogram of the target product obtained from the Comparative Example 1 of the present invention.

### EMBODIMENTS

Hereinafter, the present invention will be illustrated in detail with reference to the embodiments; however, the present invention is not limited thereto.

The raw material (R or S)N-substituent P-2-alkoxycarbonylalkyliminodiacetic acid diester used in the embodiments can be prepared according to the prior arts, while other raw materials and reagents are commercially available.

The percentages in the embodiments all refer to mass percentages, unless otherwise indicated.

A liquid chromatograph equipped with a chiral column (ES-OVS, 150 mmx4.6 mm, Agilent) is used to monitor the reaction process and product optical purity (area ratio %) and calculate the molar yield and e.e % value.

### Embodiment 1: Preparation of (S)-1-benzyl-5-oxo-piperidine-2-carboxylic acid hydrochloride (I₁)

To a 500 ml 4-neck flask equipped with a stirrer, a thermometer, and a constant-pressure dropping funnel, 100 g of tetrahydrofuran, 100g of dichloromethane, 37.9g (0.10 mol) of N-benzyl-2S-ethoxycarbonyl ethyl diethyl iminodiacetate 40.0g (0.3 mol) of aluminium trichloride are added, and then the reaction mixture is stirred well, cooled to -5-0° C., and dropwise added 15.2g (0.15 mol) of triethylamine within 1-2 hours under the same temperature. Then, the reaction mixture is stirred for reaction at the temperature of 10-15° C. for 3 hours. The reaction liquid is then poured slowly into 100 g of water and separated. The resulting organic phase is then washed once with 100g of 5 wt % sodium bicarbonate aqueous solution and separated. After the organic phase is condensed to dry, 100g of 30 wt % hydrochloric acid is added to the concentrate, stirred at 60-65° C. to react for 2 hours, and then cooled down to 20-25° C. The resulting reaction liquid is then added to the mixture of 50 g of water and 100g of dichloromethane and separated. Then, the aqueous phase is extracted by dichloromethane (50 g each), and the organic phases are combined. After the solvent is recovered through atmospheric distillation, 19.5g of (S)-1-benzyl-5-oxo-piperidine-2-carboxylic acid hydrochloride (Ii) is obtained through reduced pressure distillation of the aqueous phase.

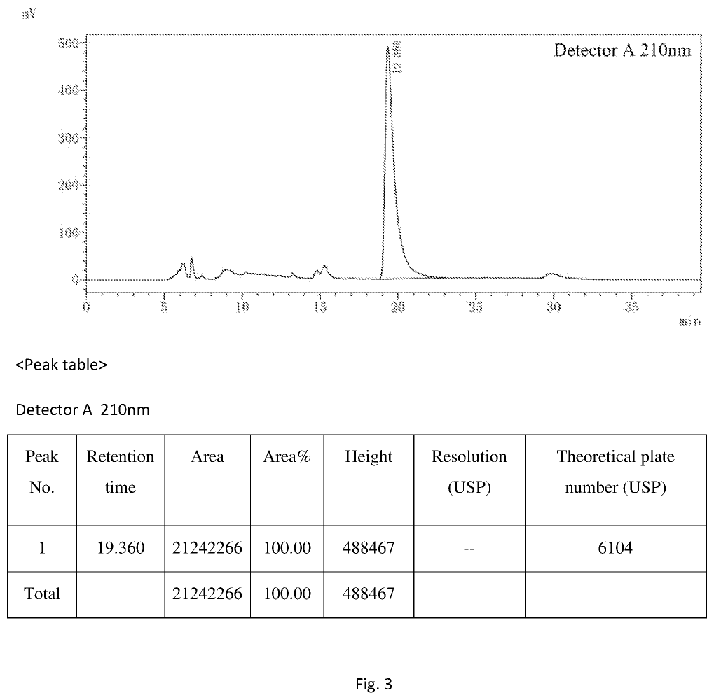

The normal-phase HPLC chromatogram of the resulting target product from the embodiment is provided in FIG. 3. According to the figure, the resulting target product from the embodiment is with an 100% optical purity and a 72.3% molar yield. The specific optical rotation of the resulting target product from the embodiment is:

[α]²⁰_{D}=−26.2° (c=0.01, water).

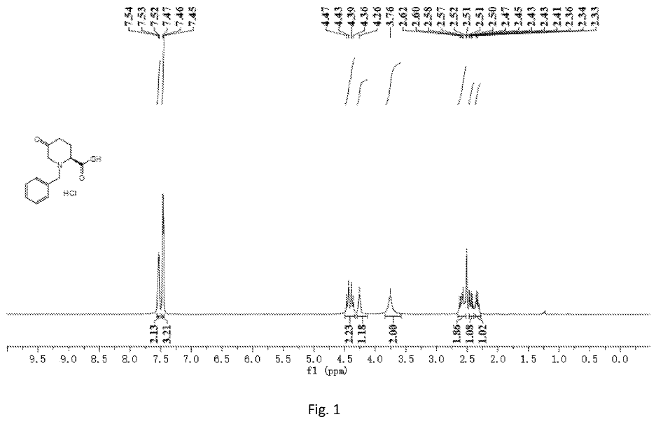

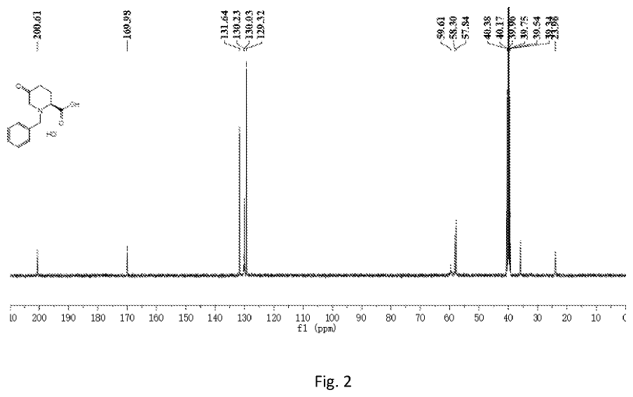

The NMR (Nuclear Magnetic Resonance) hydrogen spectrogram and NMR (Nuclear Magnetic Resonance) carbon spectrogram of the resulting target product from the embodiment are provided in FIG. 1 and FIG. 2 respectively.

NMR (Nuclear Magnetic Resonance) data of the resulting target product are provided below: ¹HNMR(400 MHz, DMSO-d₆)δ: 7.47 (m, 5H), 4.41 (dd, J=31.2, 12.9 Hz, 2H), 4.26 (s, 1H), 3.76 (s, 2H), 2.59 (m, 2H), 2.44 (m, 1H), 2.34 (m, 1H).

¹³C-NMR (100 MHz, DMSO-d6)δ: 200.61, 169.98, 131.64, 130.23, 130.03, 129.32, 58.30, 57.84, 35.81, 23.96.

### Embodiment 2: Preparation of (S)-1-benzyl-5-oxo-piperidine-2-carboxylic acid hydrochloride (I₁)

To a 500 ml 4-neck flask equipped with a stirrer, a thermometer, and a constant-pressure dropping funnel, 100 g of tetrahydrofuran, 100g of dichloromethane, and 37.9g (0.10 mol) of N-benzyl-2S-ethoxycarbonyl ethyl diethyl iminodiacetate MO are added, and 56.9g (0.3 mol) of titanium tetrachloride are dropwise added within 0.5-1h hours; then the reaction mixture is stirred well, cooled to −5-0° C., and dropwise added 15.2g (0.15 mol) of triethylamine within 1-2 hours under the same temperature. Then, the reaction mixture is stirred for reaction at the temperature of 0-5° C. for 3 hours. The reaction liquid is then poured slowly into 100 g of water and separated. The resulting organic phase is then washed once with 100g of 5 wt % sodium bicarbonate aqueous solution and separated. After the organic phase is condensed to dry, 100g of 30 wt % hydrochloric acid is added to the concentrate, stirred at 60-65° C. to react for 2 hours, and then cooled down to 20-25° C. The resulting reaction liquid is then added to the mixture of 50 g of water and 100g of dichloromethane and separated. Then, the aqueous phase is extracted by dichloromethane (50 g each), and the organic phases are combined. After the solvent is recovered through atmospheric distillation, 21.2g of (S)-1-benzyl-5-oxo-piperidine-2-carboxylic acid hydrochloride (Ii) is obtained in an optical purity of 100.0% and a molar yield of 78.6% through reduced pressure distillation of the aqueous phase.

### Embodiment 3: Preparation of (S)-1-benzyl-5-oxo-piperidine-2-ethyl formate (12)

To a 500 ml 4-neck flask equipped with a stirrer, a thermometer, and a constant-pressure dropping funnel, 100 g of tetrahydrofuran, 100g of dichloromethane, and 37.9g (0.10 mol) of N-benzyl-2S-ethoxycarbonyl ethyl diethyl iminodiacetate (II₁), and 40.0g (0.3 mol) of aluminium trichlorideare added; then, the reaction mixture is stirred well, cooled to −5-0° C., and dropwise added 15.2g (0.15 mol) of triethylamine within 1-2 hours under the same temperature. Then, the reaction mixture is stirred for reaction at the temperature of 0-5° C. for 3 hours. The reaction liquid is then poured slowly into 100 g of water and separated. The resulting organic phase is then washed once with 100g of 5 wt % sodium bicarbonate aqueous solution and separated. After the organic phase is condensed to dry, 100g of DWF and 3g of lithium chloride are added to the concentrate, stirred at 130-135° C. to react for 2 hours, and then cooled down to 20-25° C. The resulting reaction liquid is then added to the mixture of 50 g of water and 100g of dichloromethane and separated. Then, the aqueous phase is extracted by dichloromethane (50 g each), and the organic phases are combined. After the solvent is recovered through atmospheric distillation, 19.7g of (S)-1-benzyl-5-oxo-piperidine-2-ethyl formate (1₂) is obtained in an optical purity of 100.0% and a molar yield of 75.5% through reduced pressure distillation (130-140° C./1-1.5 mmHg) of the aqueous phase.

### Embodiment 4: Preparation of (R)-1-benzyl-5-oxo-piperidine-2-carboxylic acid hydrochloride (13)

To a 500 ml 4-neck flask equipped with a stirrer, a thermometer, and a constant-pressure dropping funnel, 100 g of tetrahydrofuran, 100g of dichloromethane, and 37.9g (0.10 mol) of N-benzyl-2R-ethoxycarbonyl ethyl diethyl iminodiacetate (II₂) are added, and 56.9g (0.3 mol) of titanium tetrachloride are dropwise added within 1-1.5 hours; then the reaction mixture is stirred well, cooled to −5-0° C., and dropwise added 15.2g (0.15 mol) of triethylamine within 1-2 hours under the same temperature. Then, the reaction mixture is stirred for reaction at the temperature of 0˜5° C. for 3 hours. The reaction liquid is then poured slowly into 100 g of water and separated. The resulting organic phase is then washed once with 100g of 5 wt % sodium bicarbonate aqueous solution and separated. After the organic phase is condensed to dry, 100g of 30 wt % hydrochloric acid is added to the concentrate, stirred at 60-65° C. to react for 2 hours, and then cooled down to 20-25° C. The resulting reaction liquid is then added to the mixture of 50 g of water and 100g of dichloromethane and separated. Then, the aqueous phase is extracted by dichloromethane (50 g each), and the organic phases are combined. After the solvent is recovered through atmospheric distillation, 21.1 of (R)-1-benzyl-5-oxo-piperidine-2-carboxylic acid hydrochloride (1₃) is obtained in an optical purity of 100.0% and a molar yield of 78.2% through reduced pressure distillation of the aqueous phase. The specific optical rotation is: [α]²⁰_{D}=+26.2° (c=0.01, water).

### Embodiment 5: Preparation of (S)-1-benzyloxycarbonyl-4-oxo-tetrahydropyrrole-2-carboxylic acid hydrobromide (14)

To a 500 ml 4-neck flask equipped with a stirrer, a thermometer, and a constant-pressure dropping funnel, 350g of dichloromethane and 40.9g (0.10 mol) of N-benzyloxycarbonyl-2S-ethoxycarbonyl methyl diethyl iminodiacetate (II₃) are added, and 56.9g (0.3 mol) of titanium tetrachloride are dropwise added within 0.5-1 hours; then the reaction mixture is stirred for 2 hours, cooled to −10-0° C., and dropwise added 30.3 (0.3 mol) of triethylamine within 1-2 hours under the same temperature. Then, the reaction mixture is stirred for reaction at the temperature of 0-5° C. for 3 hours, quenched by adding water, and separated. After the organic phase is condensed to dry, 100g of 40 wt % hydrobromic acid is added to the concentrate, stirred at 60-65° C. to react for 2 hours, and then cooled down to 20-25° C. The resulting reaction liquid is then added to the mixture of 50 g of water and 100g of dichloromethane and separated. Then, the aqueous phase is extracted by dichloromethane (50 g each), and the organic phases are combined. After the solvent is recovered through atmospheric distillation, 23.4g of (S)-1-benzyloxycarbonyl-4-oxo-tetrahydropyrrole-2-carboxylic acid hydrobromide (I4) is obtained in an optical purity of 100.0% and a molar yield of 78.6%. The specific optical rotation is: [a]²⁰_{D}=18.3° (c=0.01, dichloromethane).

NMR (Nuclear Magnetic Resonance) data of the resulting target product are provided below: 1H NMR (400 MHz, DMSO-d6) δ: 7.47 (m, 5H), 4.68 (s, 2H), 3.70 (m, 1H,), 3.43 (d, J=16.8 Hz, 1H), 3.15 (d, J=16.8 Hz, 1H), 2.75 (m, 1H), 2.63 (m, 1H).

### Embodiment 6: Preparation of (S)-1-p-chlorobenzyl-5-oxo-piperidine-2-carboxylic acid hydrochloride (16)

To a 500 ml 4-neck flask equipped with a stirrer, a thermometer, and a constant-pressure dropping funnel, 100 g of tetrahydrofuran, 100g of dichloromethane, 45.6g (0.10 mol) of N-p-chlorobenzyl-2S-isopropoxycarbonyl ethyl diethyl iminodiisopropyladipate (II₄), and 40.0g (0.3 mol) of aluminium trichloride are added, and then the reaction mixture is stirred well, cooled to −5-0° C., and dropwise added 15.2g (0.15 mol) of triethylamine within 1-2 hours under the same temperature. Then, the reaction mixture is stirred for reaction at the temperature of 10-15° C. for 3 hours. The reaction liquid is then poured slowly into 100 g of water and separated. The resulting organic phase is then washed once with 100g of 5 wt % sodium bicarbonate aqueous solution and separated. After the organic phase is condensed to dry, 100g of 30 wt % hydrochloric acid is added to the concentrate, stirred at 60-65° C. to react for 2 hours, and then cooled down to 20-25° C. The resulting reaction liquid is then added to the mixture of 50 g of water and 100g of dichloromethane and separated. Then, the aqueous phase is extracted by dichloromethane (50 g each), and the organic phases are combined. After the solvent is recovered through atmospheric distillation, 22.3g of (S)-1-p-chlorobenzyl-5-oxo-piperidine-2-carboxylic acid hydrochloride (16) is obtained in a molar ratio of 73.2% through reduced pressure distillation of the aqueous phase.

NMR (Nuclear Magnetic Resonance) data of the resulting target product are provided below:

¹HNMR(400 MHz, DMSO-d6)δ: 7.51 (d, 2H), 7.43 (d, 2H), 4.41 (dd, J=31.2, 12.9 Hz, 2H), 4.26 (s, 1H), 3.76 (s, 2H), 2.59 (m, 2H), 2.44 (m, 1H), 2.34 (m, 1H).

### Comparative Example 1: Preperation of (R,S)-1-benzyl-5-oxo-piperidine-2-carboxylic acid hydrochloride (I₅)

To a 500 ml 4-neck flask equipped with a stirrer, a thermometer, and a constant-pressure dropping funnel, 200 g of tetrahydrofuran and 10.8g (0.2 mol) of sodium methoxide are added, and then the reaction mixture is stirred well, cooled to −5-0° C., and dropwise added 37.9g (0.10 mol) of N-benzyl-2S-ethoxycarbonyl ethyl diethyl iminodiacetate (II₁) within 1-2 hours under the same temperature. Then, the reaction mixture is stirred for reaction at the temperature of 0-5° C.for 3 hours. The reaction liquid is then poured slowly into the mixture of 100 g of water and 100g of dichloromethane and then separated. Then, the resulting aqueous phase is extracted by dichloromethane (50 g each), and the organic phases are combined. After the organic phase is condensed to dry, 100g of 30 wt % hydrochloric acid is added to the concentrate, stirred at 60-65° C. to react for 2 hours, and then cooled down to 20-25° C. The resulting reaction liquid is then added to the mixture of 50 g of water and 100g of dichloromethane and separated. Then, the aqueous phase is extracted by dichloromethane (50 g each), and the organic phases are combined. After the solvent is recovered through atmospheric distillation, 20.1g of (R,S)-1-benzyl-5-oxo-piperidine-2-carboxylic acid hydrochloride (I₅) is obtained through reduced pressure distillation of the aqueous phase.

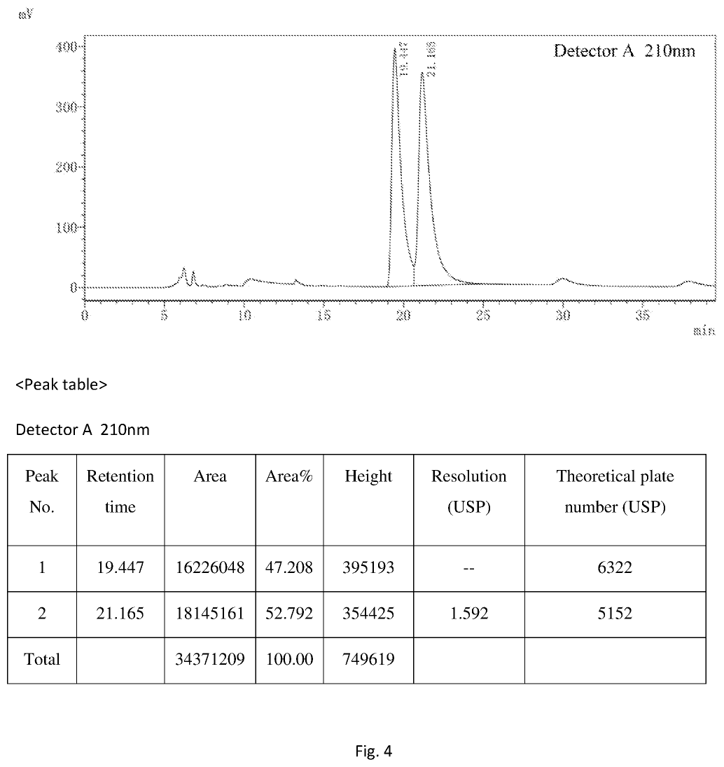

The normal-phase HPLC chromatogram of the resulting target product from the comparative example is provided in FIG. 4. According to the figure, the resulting product is with a 0.0% optical purity and a 74.6% molar yield. The specific optical rotation of the resulting product is: [α]²⁰_{D} =0 ^{°} (c=0.01, water).

According to Comparative Example 1, when cyclized under strong base conditions, the chiral carbon atom of the N-benzyl-2S-ethoxycarbonyl ethyl diethyl iminodiacetate is racemized, which further indicates that the reaction conditions are not suitable for the preparation of high photoactive target compounds.

### Comparative Example 2: Preparation of (S)-1-benzyl-5-oxo-piperidine-2-carboxylic acid hydrochloride (I₁)

To a 500 ml 4-neck flask equipped with a stirrer, a thermometer, and a constant-pressure dropping funnel, 200 g of tetrahydrofuran, 37.9g (0.10 mol) of N-benzyl-2S-ethoxycarbonyl ethyl diethyl iminodiacetate (II₁), and 40.0g (0.3 mol) of aluminium trichloride are added, and then the reaction mixture is stirred well, cooled to −5-0° C., and dropwise added 15.2g (0.15 mol) of triethylamine within 1-2 hours under the same temperature. Then, the reaction mixture is stirred for reaction at the temperature of 5560° C.for 3 hours. The reaction liquid is then poured slowly into 100 g of water and separated. The resulting organic phase is then washed once with 100g of 5 wt % sodium bicarbonate aqueous solution and separated. After the organic phase is condensed to dry, 100g of 30 wt % hydrochloric acid is added to the concentrate, stirred at 60-65° C. to react for 2 hours, and then cooled down to 20-25° C. The resulting reaction liquid is then added to the mixture of 50 g of water and 100g of dichloromethane and separated. Then, the aqueous phase is extracted by dichloromethane (50 g each), and the organic phases are combined. After the solvent is recovered through atmospheric distillation, 9.8g of (S)-1-benzyl-5-oxo-piperidine-2-carboxylic acid hydrochloride (Ii) is obtained in an optical purity of 96.3% and a molar yield of 56.7%% through reduced pressure distillation of the aqueous phase.

According to Comparative Example 2, too high a cyclization reaction temperature will reduce the yield, which is because that the N-benzyl-2S-ethoxycarbonyl ethyl diethyl iminodiacetate is subjected to polymerization under high temperatures, and also result in a lower optical purity of the product.

What is claimed is:

1. A preparation method of a chiral oxo-aza-cycloalkane compound, comprising the steps of:
a compound of formula II is cyclized and then decarboxylated to obtain a compound of formula I or the salt thereof; 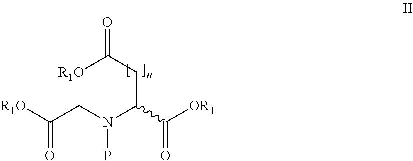
wherein, the substituent R₁ in the compound of formula II is methyl, ethyl, n-propyl, isopropyl, tert-butyl, n-butyl, sec-butyl or benzyl; the substituent P is benzyl, o-methoxybenzyl, m-methoxybenzyl, p-methoxybenzyl, 2,4-dimethoxybenzyl, o-methylbenzyl, m-methylbenzyl, p-methylbenzyl, o-chlorobenzyl, p-chlorobenzyl, m-chlorobenzyl, benzoyl, methoxycarbonyl, tert-butoxycarbonyl, or benzyloxycarbonyl; n is 1, 2, or 3.

2. The preparation method of a chiral oxo-aza-cycloalkane compound according to claim 1, wherein the compound of formula II is N-benzyl-2S-methoxycarbonyl methyl dimethyl iminodiacetate, N-benzyl-2S-ethoxycarbonyl methyl diethyl iminodiacetate, N-benzyl-2S-methoxycarbonyl ethyl dimethyl iminodiacetate, N-benzyl-2S-ethoxycarbonyl ethyl diethyl iminodiacetate, N-benzyl-2R-methoxycarbonyl ethyl dimethyl iminodiacetate, N-benzyl-2R-ethoxycarbonyl ethyl diethyl iminodiacetate, N-benzyl-2S-methoxycarbonyl propyl dimethyl iminodiacetate, N-benzyl-2S-ethoxycarbonyl propyl diethyl iminodiacetate, N-p-chlorobenzyl-2S-Isopropoxycarbonyl ethyl diethyl iminodiisopropyladipate, or N-benzyloxycarbonyl-2S-ethoxycarbonyl methyl diethyl iminodiacetate.

3. The preparation method of a chiral oxo-aza-cycloalkane compound according to claim 1, wherein the compound of formula II is cyclized in a solvent under the action of a cyclization reagent.

4. The preparation method of a chiral oxo-aza-cycloalkane compound according to claim 3, wherein the solvent is tetrahydrofuran, 2-methyl tetrahydrofuran, 1,4-dioxane, ethylene glycol dimethyl ether, methyl tert-butyl ether, methoxy-cyclopentane, dichloromethane, chloroform, 1,2-dichloroethane, normal hexane, n-heptane, or methylbenzene, or a combination of two or more thereof.

5. The preparation method of a chiral oxo-aza-cycloalkane compound according to claim 3, wherein the cyclization reagent is the mixture of a lewis acid and a lewis base.

6. The preparation method of a chiral oxo-aza-cycloalkane compound according to claim 5, wherein the lewis acid is aluminium trichloride, boron trifluoride, titanium tetrachloride, or stannic chloride.

7. The preparation method of a chiral oxo-aza-cycloalkane compound according to claim 5, wherein the lewis base is trimethylamine, triethylamine, tri-n-butylamine, diisopropylethylamine, or pyridine, or a combination of two or more thereof.

8. The preparation method of a chiral oxo-aza-cycloalkane compound according to claim 5, wherein the molar ratio among the lewis acid, lewis base, and compound of formula II is (1.0-4.0): (1.0-4.0):1.

9. The preparation method of a chiral oxo-aza-cycloalkane compound according to claim 3, wherein one or more of the following conditions are selected:
a. the mass ratio between the solvent and the compound of formula II is (3-10):1; and
b. the temperature of the cyclization reaction is -60-50° C.; preferably, the temperature of the cyclization reaction is -30-20° C.; more preferably, the temperature of the cyclization reaction is 0-15° C.

10. The preparation method of a chiral oxo-aza-cycloalkane compound according to claim 3, wherein one or more of the following conditions are selected:
a. after the cyclization reaction, the compound of formula II proceeds directly to the next step without separation; and
b. the decarboxylation reaction is to obtain the compound of formula I through thermal decarboxylation in the presence of N, N-dimethyl formamide (DMF) and lithium chloride, or obtain a salt of the compound of formula I through hydrolysis decarboxylation in the presence of an acid.

11. The preparation method of a chiral oxo-aza-cycloalkane compound according to claim 10, wherein one or more of the following conditions are selected:
a. the mass ratio between the DMF and the compound of formula II is (2.0-10.0): 1;
preferably, the mass ratio between the DMF and the compound of formula II is (2.0-5.0): 1;
b. the mass ratio between the lithium chloride and the compound of formula II is (2.0-20.0%):1; preferably, the mass ratio between the lithium chloride and the compound of formula II is (5.0-10.0%):1;
c. the temperature of the thermal decarboxylation reaction is 100-180° C.; preferably, the temperature of the thermal decarboxylation reaction is 130-150° C.;
d. the acid is hydrochloric acid, hydrobromic acid, hydroiodic acid, acetic acid, sulfuric acid, or phosphoric acid, or a combination of any two or more thereof;
e. the molar ratio between the acid and the compound of formulaII is (1.0-10.0):1; and
f. the temperature of the hydrolysis decarboxylation reaction is 30-110° C.; preferably, the temperature of the hydrolysis decarboxylation reaction is 60-100° C.; more preferably, the temperature of the hydrolysis decarboxylation reaction is 60-80° C.

12. The preparation method of a chiral oxo-aza-cycloalkane compound according to claim 1, wherein the salt of the compound of formula I is the hydrochloride, hydrobromide, hydriodate, sulphate, phosphate, or acetate of the compound of formula I.

13. The preparation method of a chiral oxo-aza-cycloalkane compound according to claim 1, wherein the reaction route is as shown below: 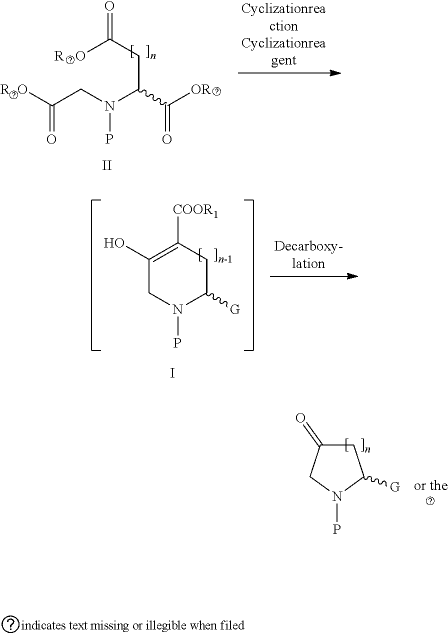
wherein, the substituent R₁ in the structural formula of the compound of formula II is methyl, ethyl, n-propyl, isopropyl, tert-butyl, n-butyl, sec-butyl, or benzyl; the substituent P is benzyl, o-methoxybenzyl, m-methoxybenzyl, p-methoxybenzyl, 2,4-dimethoxybenzyl, o-methylbenzyl, m-methylbenzyl, p-methylbenzyl, o-chlorobenzyl, p-chlorobenzyl, m-chlorobenzyl, benzoyl, methoxycarbonyl, tert-butoxycarbonyl, or benzyloxycarbonyl; n is 1, 2 or 3; and
in the intermediate obtained through the cyclization reaction of the compound of formula II, the n, substituent P, and substituent R₁ have the same meanings as those in the structural formula of the compound of formula II; when n is 1, it means that the double-bonded carbon linked with COOR₁ is directly bonded to the carbon linked with the substituent G; the substituent G is COOH or COOR, wherein R is methyl, ethyl, n-propyl, isopropyl, tert-butyl, n-butyl, sec-butyl, or benzyl and the substituent R has the same meaning as the substituent R₁ in the structural formula of the compound of formula II. In the structural formula of the compound of formula I, the substituent P, n, and substituent G have the same meanings as those in the above intermediate.

14. The preparation method of a chiral oxo-aza-cycloalkane compound according to claim 1,
wherein the compound of formula I is:
(S)-1-benzyl-5-oxo-piperidine-2-carboxylic acid hydrochloride (I₁),
(S)-1-benzyl-5-oxo-piperidine-2-ethyl formate (12),
(R)-1-benzyl-5-oxo-piperidine-2-carboxylic acid hydrochloride (I₃),
(S)-1-benzyloxycarbonyl-4-oxo-tetrahydropyrrole-2-carboxylic acid hydrobromide (I₄),
or (S)-1-P-chlorobenzyl-5-oxo-piperidine-2-carboxylic acid hydrochloride (16).
